(19) United States
(12) Patent Application Publication
(10) Pub. No.: US 2019/0133425 A1
(43) Pub. Date: May 9, 2019

(54) ENDOSCOPE SYSTEM, TERMINAL DEVICE, SERVER, AND TRANSMISSION METHOD

(71) Applicant: OLYMPUS CORPORATION, Tokyo (JP)

(72) Inventors: Katsuyoshi TANIGUCHI, Tokyo (JP); Yuta MAKINO, Tokyo (JP)

(73) Assignee: OLYMPUS CORPORATION, Tokyo (JP)

(21) Appl. No.: 16/242,284

(22) Filed: Jan. 8, 2019

Related U.S. Application Data
(63) Continuation of application No. PCT/JP2017/036016, filed on Oct. 3, 2017.

(30) Foreign Application Priority Data
Oct. 20, 2016 (JP) 2016-205896

(51) Int. Cl. A61B 1/045; A61B 1/05; A61B 1/00

(52) U.S. Cl. CPC A61B 1/045; A61B 1/05; G02B 23/24; A61B 1/00009; A61B 1/00011

(57) ABSTRACT

An endoscope system including: a server to accumulate image data groups and status information including operation history of observation; a terminal device to allow selecting image data groups accumulated in the server; and a processor provided in the server or the terminal device, wherein the processor is configured to execute: estimating a type of an observation workflow that differs depending on whether observation operation has been performed on the selected image data groups or whether the performed observation operation has been completed, the estimation being performed based on status information corresponding to the selected image data groups; setting transmission image data groups to be transmitted from the server to the terminal device among the selected image data groups based on the estimated type of the observation workflow; and performing control of transmitting the set transmission image data groups from the server to the terminal device.

### CROSS-REFERENCE TO RELATED APPLICATION

This application is a continuation of PCT international application Ser. No. PCT/JP2017/036016, filed on Oct. 3, 2017, which designates the United States, incorporated herein by reference, and which claims the benefit of priority from Japanese Patent Application No. 2016-205896, filed on Oct. 20, 2016, incorporated herein by reference.

### BACKGROUND

The present disclosure relates to an endoscope system, a terminal device, a server, a transmission method, and a program for transmitting in-vivo image data groups to a client terminal device.

In the field of endoscopes, examinations using a capsule endoscope that is introduced into a subject and captures in-vivo images have been known. The capsule endoscope is a device having an imaging function, a wireless function, and the like inside a capsule-shaped casing with a size that is introducible into digestive tracts of a subject, and after being swallowed from the mouth of the subject, generates image data by sequentially performing imaging while moving in the digestive tracts by peristaltic movement and the like. The generated image data is wirelessly transmitted in sequence from the capsule endoscope to outside of the subject.

The image data wirelessly transmitted from the capsule endoscope is received by a receiving device that is installed outside the subject, and accumulated in a recording medium, such as a built-in memory incorporated in the receiving device or a removable portable memory. After completion of an examination, the image data accumulated in the recording medium is transferred (downloaded) to an information management apparatus, such as a workstation, and subjected to predetermined image processing. A medical worker diagnoses the subject by observing images corresponding to the image data that has been subjected to the image processing.

Meanwhile, in recent years, construction of network systems for managing medical information, such as image data and examination information, has been in progress. By transmitting and receiving various kinds of medical information including medical image data to and from the network systems as described above via a network, various attempts, such as an attempt to centrally control medical images in a single facility and an attempt to allow a resident specialized doctor who works in a facility that is different from a facility in which an examination has been conducted to observe medical images, have been made.

For example, an image transfer system including a server that records an image data group that is captured by each of a CT and an MRI and a terminal device that is communicably connected to the server and displays images corresponding to image data transmitted from the server has been known (for example, Japanese Laid-open Patent Publication No. 2009-230755). In this technology, when a diagnosis is made by comparing a first image group captured by the CT and a second image group captured by the MRI, and if the terminal device is displaying the first image group captured by the CT, the server determines an image transfer order of the second image group on the basis of an image that is being displayed on the terminal device, and sequentially transmits the second image group to the terminal device in the determined image transfer order.

### SUMMARY

An endoscope system according to one aspect of the present disclosure includes: a server configured to accumulate image data groups and status information that includes operation history of observation corresponding to each of the image data groups; a terminal device configured to allow selecting any of the image data groups accumulated in the server via a network, the terminal device allowing browsing the selected image data groups; and a processor comprising hardware that is provided in the server or the terminal device, wherein the processor is configured to execute: estimating a type of an observation workflow that differs depending on whether observation operation has been performed on the selected image data groups or whether the performed observation operation has been completed, the estimation being performed on the basis of status information corresponding to the selected image data groups; setting transmission image data groups being images to be transmitted from the server to the terminal device among the selected image data groups on the basis of the estimated type of the observation workflow; and performing control of transmitting the set transmission image data groups from the server to the terminal device.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of an endoscope system according to a first embodiment;

FIG. 2 is a block diagram illustrating a functional configuration of a server according to the first embodiment;

FIG. 3 is a block diagram illustrating a functional configuration of a terminal device according to the first embodiment;

FIG. 4 is a diagram illustrating an example of an observation screen displayed by the terminal device according to the first embodiment;

FIG. 5 is a flowchart illustrating the outline of a process performed by the terminal device according to the first embodiment;

FIG. 6A is a diagram schematically illustrating a transmission order of image data groups of a case file that is transmitted to the terminal device in a new observation workflow and captured images;

FIG. 6B is a diagram schematically illustrating image data groups of a case file that is transmitted to the terminal device in an observation completed examination workflow, a transmission order of the image data groups, and images captured in the new observation workflow;

FIG. 7A is a diagram schematically illustrating a transmission order of image data groups of a case file that is transmitted to the terminal device in the new observation workflow;

FIG. 7B is a diagram schematically illustrating image data groups of a case file that is transmitted to the terminal device in the observation completed examination workflow and a transmission order;

FIG. 8 is a flowchart illustrating the outline of a process performed by a terminal device according to a second embodiment;

FIG. 9 is a flowchart illustrating the outline of a process performed by a terminal device according to a third embodiment;

FIG. 10A is a diagram schematically illustrating a transmission order of image data groups of a case file that is transmitted to a terminal device in the new observation workflow, images that are captured in the new observation workflow, and a browsing time for each predetermined period;

FIG. 10B is a diagram schematically illustrating image data groups of a case file that is transmitted to the terminal device in the observation completed examination workflow, a transmission order of the image data groups, and images that are captured in the new observation workflow;

FIG. 11 is a flowchart illustrating the outline of a process performed by a terminal device according to a modification of the third embodiment;

FIG. 12 is a block diagram illustrating a functional configuration of a server according to a fourth embodiment; and

FIG. 13 is a flowchart illustrating the outline of a process performed by the server according to the fourth embodiment.

### DETAILED DESCRIPTION

Embodiments of an endoscope system according to the present disclosure will be described below with reference to the drawings. The present disclosure is not limited by the embodiments below. In the description of the drawings, the same components are denoted by the same reference signs, and explanation thereof will be omitted.

### First Embodiment

Configuration of Endoscope System

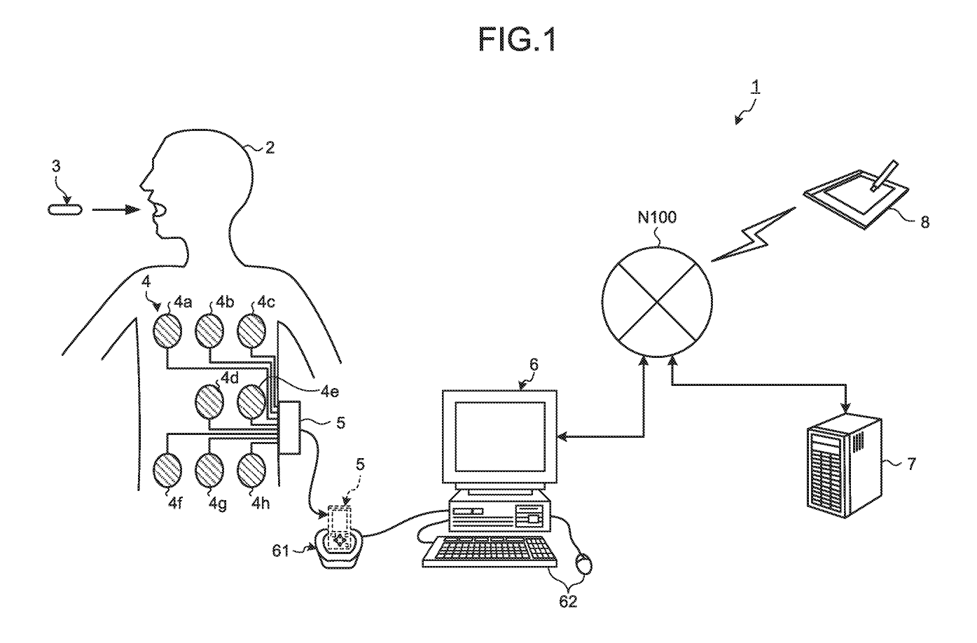

FIG. 1 is a schematic diagram illustrating a schematic configuration of an endoscope system according to a first embodiment. An endoscope system 1 illustrated in FIG. 1 includes a capsule endoscope 3 that is introduced into a subject 2, generates image data by capturing in-vivo images of the subject 2, and transmits the images to outside by superimposing the images on wireless signals; a receiving device 5 that receives the wireless signals transmitted from the capsule endoscope 3 via a receiving antenna unit 4 mounted on the subject 2; an examination information management apparatus 6 (workstation) that manages examination information on an examination performed on the subject 2 and image data groups that are examination results, performs image processing on the image data, and displays images corresponding to the image data; a server 7 that records the examination information and the image data groups that are transmitted from the examination information management apparatus 6 via a network N100 for each case file, and transmits image data groups of a case file requested by a terminal device 8; and the terminal device 8 (client) that acquires the examination information and the image data groups of the case file from outside via the network N100. The examination information management apparatus 6, the server 7, and the terminal device 8 are connected to one another via the network N100 in a wired or wireless manner so as to be able to perform bi-directional communication with one another.

The capsule endoscope 3 has an imaging function to capture in-vivo images of the subject 2, and a wireless communication function to transmit, to the receiving antenna unit 4, in-vivo information including image data that is obtained by capturing in-vivo images of the subject 2. The capsule endoscope 3 passes through the esophagus of the subject 2 by being swallowed by the subject 2, and moves inside the body cavity of the subject 2 by peristaltic movement of the digestive tract lumen. The capsule endoscope 3 sequentially captures images inside the body cavity of the subject 2 at a very short time interval, such as a 0.5-second interval (2 fps), while moving inside the body cavity of the subject 2, generates image data of the captured in-vivo images of the subject 2, and sequentially transmits the image data to the receiving antenna unit 4.

The receiving antenna unit 4 includes receiving antennas 4a to 4h. The receiving antennas 4a to 4h receive wireless signals from the capsule endoscope 3 and transmit the wireless signals to the receiving device 5. The receiving antennas 4a to 4h are constructed using loop antennas, active antennas, or the like. Each of the receiving antennas 4a to 4h is mounted on a predetermined position on an outer surface of the subject 2, such as a position corresponding to each of organs of the subject 2 through which the capsule endoscope 3 passes.

The receiving device 5 records in-vivo image data of the subject 2 included in the wireless signals that are transmitted from the capsule endoscope 3 via the receiving antennas 4a to 4h, or display images corresponding to the in-vivo image data of the subject 2. The receiving device 5 records positional information on the capsule endoscope 3, time information indicating a time, and the like via the receiving antennas 4a to 4h, and records them in association with image data. The receiving device 5 is housed in a receiving device holder (not illustrated) and carried by the subject 2 while an examination using the capsule endoscope 3 is being performed, e.g., during a period from introduction of the capsule endoscope 3 through the mouth of the subject 2 to discharge of the capsule endoscope 3 from the subject 2 with movement through the digestive tracts. The receiving device 5 is removed from the subject 2 after the examination using the capsule endoscope 3 is completed, and connected to the examination information management apparatus 6 to transfer image data groups that are received from the capsule endoscope 3. Further, after the examination using the capsule endoscope 3 is completed, the receiving device 5 may transmit image data to the server 7 via the network N100 without via the examination information management apparatus 6 to be described later, or it may be possible to transfer or move image data using a recording medium (not illustrated), such as a memory card, that is detachably attachable to the

The examination information management apparatus 6 displays images corresponding to the image data of the subject 2 acquired via the receiving device 5, and records the image data and examination information on the subject 2 in an associated manner. The examination information management apparatus 6 includes a cradle 61 that reads image data or the like from the receiving device 5, and an operation input device 62, such as a keyboard and a mouse. When the receiving device 5 is attached, the cradle 61 acquires, from the receiving device 5, image data and related information, such as positional information, time information, identification information on the capsule endoscope 3, and subject information on the subject 2, which are associated with the image data, and transfers the various kinds of acquired information to the examination information management apparatus 6. The operation input device 62 receives input of operation performed by a user. The user observes a living body site, such as an esophagus, a stomach, a small intestine, and a large intestine, of the subject 2 to make a diagnosis of a lesion or the like of the subject 2 and perform observation by viewing images of the subject 2 displayed by the examination information management apparatus 6 while operating the operation input device 62. In addition, the examination information management apparatus 6 transmits the image data and the related information acquired from the receiving device 5 to the server 7 via the network N100 in an associated manner. The examination information management apparatus 6 is constructed using a central processing unit (CPU), a display panel made of liquid crystal, organic electro luminescence (EL), or the like, and a recording medium, such as a volatile memory or a non-volatile memory.

The server 7 records the image data groups and the related information that are transmitted from the examination information management apparatus 6 via the network N100 for each case file, in an associated manner. Here, the related information is identification information for identifying the subject 2, capsule identification information for identifying the capsule endoscope 3 that is introduced into the subject 2, facility information for identifying a hospital or a facility in which the subject 2 is diagnosed, doctor identification information for identifying a doctor who diagnoses the subject 2, and the like. Further, when the terminal device 8 issues a request to transmit image data groups of the case file via the network N100, the server 7 transmits the image data groups of the case file requested by the terminal device 8 in a predetermined transmission order. A detailed configuration of the server 7 will be described later.

The terminal device 8 displays a list of image data groups of a plurality of case files recorded in the server 7 via the network N100, and requests the server 7 to transmit image data groups of a case file that is selected by a user. The terminal device 8 displays images corresponding to image data transmitted from the server 7 via the network N100, and transmits operation information that the terminal device 8 has received to the server 7. A detailed configuration of the terminal device 8 will be described in detail later.

Detailed Configuration of Server

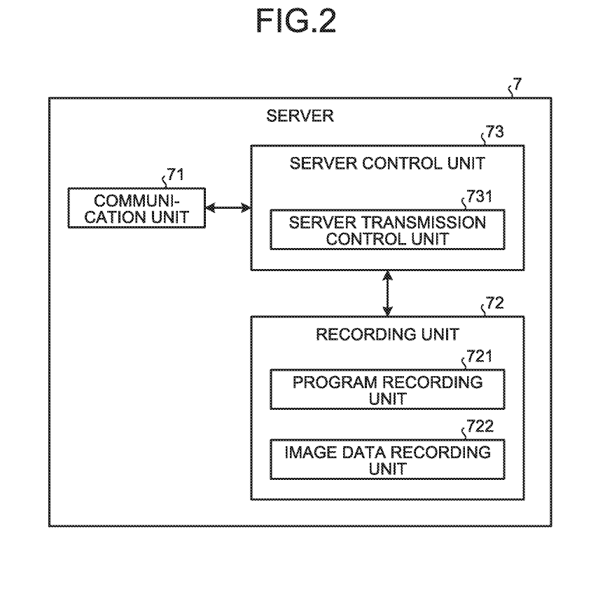

First, the detailed configuration of the server 7 will be described. FIG. 2 is a block diagram illustrating a functional configuration of the server 7. The server 7 illustrated in FIG. 2 includes a communication unit 71, a recording unit 72, and a server control unit 73. Meanwhile, in the first embodiment, the server 7 functions as an accumulating unit.

The communication unit 71 transmits and receives various kinds of data including information that is needed for communication with an external apparatus, such as the examination information management apparatus 6 and the terminal device 8, via the network N100, in accordance with a predetermined protocol under the control of the server control unit 73.

The recording unit 72 is constructed using a recording medium, such as a Flash memory, a RAM, or an HDD, and records various kinds of information related to the server 7 and image data and various kinds of information that are received via the communication unit 71. The recording unit 72 includes a program recording unit 721 that records a program for operating the server 7 and programs for various applications that are executed on the terminal device in response to a request from the terminal device 8, and an image data recording unit 722 that records an image data group and the like generated by the capsule endoscope 3. Here, the image data group is any of a plurality of pieces of chronologically successive image data, a plurality of pieces of image data captured at predetermined intervals, and a plurality of pieces of picked-up image data.

The server control unit 73 comprehensively controls operation of the server 7 by transferring instructions and data to each of the units included in the server 7. The server control unit 73 includes a server transmission control unit 731.

The server transmission control unit 731 transmits image data groups of case files that are recorded in the image data recording unit 722, in response to a request signal transmitted from the terminal device 8.

Detailed Configuration of Terminal Device

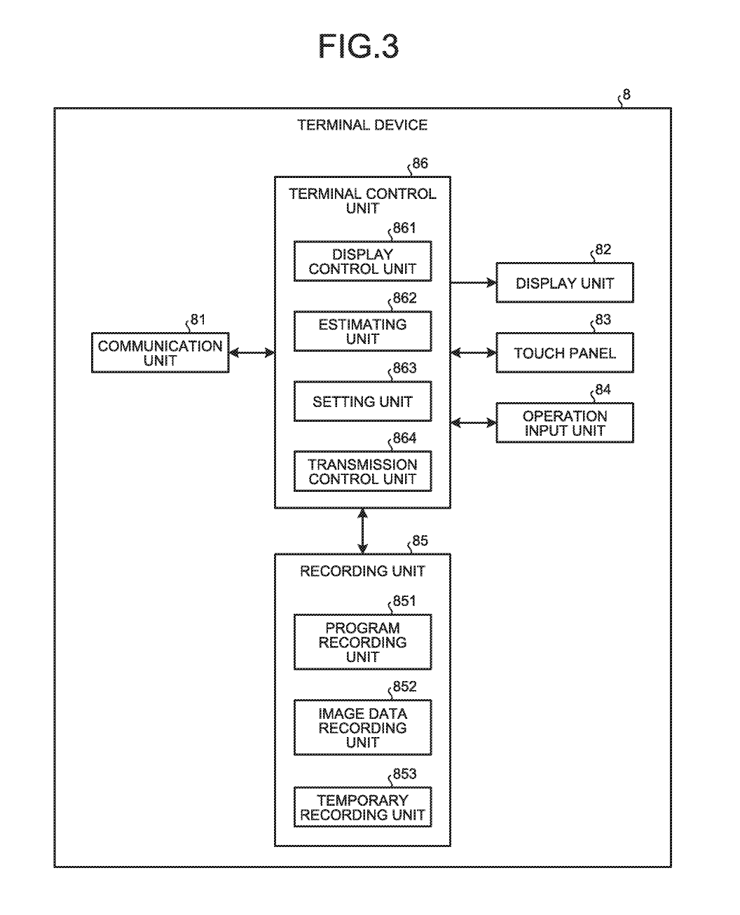

Next, the detailed configuration of the terminal device 8 will be described. FIG. 3 is a block diagram illustrating a functional configuration of the terminal device 8. The terminal device 8 illustrated in FIG. 3 includes a communication unit 81, a display unit 82, a touch panel 83, an operation input unit 84, a recording unit 85, and a terminal control unit 86. In the first embodiment, the terminal device 8 functions as a browser unit. While an example will be described in which the terminal device 8 is a portable multi-function tablet in the first embodiment, the embodiments are not limited to this example. For example, it is possible to apply a notebook personal computer and it is of course possible to apply a mobile phone, such as a smartphone.

The communication unit 81 transmits and receives wireless signals of various kinds of data including information that is needed for communication with the server 7 via the network N100, in accordance with a predetermined protocol under the control of the terminal control unit 86. Specifically, the communication unit 81 receives (downloads) an image file, in which image data of the subject 2 generated by the capsule endoscope 3 is stored, from the server 7 via the network N100, and transmits (uploads) various kinds of operation information received by the terminal device 8 to the server 7. The communication unit 81 transmits various kinds of data by any of the following communication systems: wireless fidelity (Wi-Fi) (registered trademark) communication; Bluetooth (registered trademark) communication; communication using 4G wireless or 3G wireless; and Worldwide Interoperability for Microwave Access (WiMAX) communication.

The display unit 82 displays images corresponding to image data transmitted from the server 7, various kinds of information related to the terminal device 8, or information related to various applications that are executable on the terminal device 8. The display unit 82 is constructed using a display panel made of liquid crystal, organic EL, or the like.

The touch panel 83 is provided in an overlaid manner on a display screen of the display unit 82, detects a contact position (coordinate information) touched by an external object (touch pen or a finger), and receives input of signals corresponding to a detection result. As a system of the touch panel 83, any of the resistive system, the capacitive system, and the optical system is applicable.

The operation input unit 84 receives input of signals of various kinds of operation on the terminal device 8. For example, the operation input unit 84 is constructed using a power supply button for receiving input of a signal for turning on or off a power supply of the terminal device 8, a switch button for receiving input of a signal for switching an operation mode of the display unit 82, an application execution button for receiving input of an instruction signal for executing various applications, and the like.

The recording unit 85 is constructed using a Flash memory, a random access memory (RAM), a recording medium, and the like, and records various kinds of information related to the terminal device 8. The recording unit 85 includes a program recording unit 851 for recording a program for operating the terminal device 8 and programs for executing various applications, an image data recording unit 852 for recording image data, and a temporary recording unit 853 for temporarily recording image data groups transmitted from the server 7, various kinds of information being processed by the terminal device 8, and the like.

The terminal control unit 86 controls operation of the terminal device 8 by transferring instructions and data to each of the units included in the terminal device 8. The terminal control unit 86 is constructed using a CPU or the like. The terminal control unit 86 includes a display control unit 861, an estimating unit 862, a setting unit 863, and a transmission control unit 864.

The display control unit 861 controls a display mode of the display unit 82. The display control unit 861 causes the display unit 82 to display images corresponding to image data that is received from the server 7 via the network N100.

The estimating unit 862 estimates a type of an observation workflow for browsing image data groups on the terminal device 8, on the basis of status information indicating a status of image data groups that are selected, via a touch panel 83 or the operation input unit 84, from among image data groups that are recorded in the server 7. Here, the status information includes presence/absence information related to presence or absence of each of finding information that is related to findings of a user on the image data groups, diagnosis information that is based on an observation result on the image data groups, and signature information that is related to a signature of the user who has given the diagnosis information. The estimating unit 862 estimates an observation workflow as a new observation workflow for performing new observation on the image data groups when the finding information is absent, estimates the observation workflow as a suspended observation resume workflow for suspending observation on the image data groups and thereafter resuming the observation when the finding information is present and each of the diagnosis information and the signature information is absent, and estimates the observation workflow as an observation completed examination workflow for performing observation again after observation is completed when all of the finding information, the diagnosis information, and the signature information are present.

The setting unit 863 sets transmission image data groups that are to be transmitted to the terminal device 8 among the image data groups that are selected by the terminal device 8, on the basis of the observation workflow estimated by the estimating unit 862. Further, the setting unit 863 sets a transmission order to transmit, to the terminal device 8, the image data groups that are selected in the terminal device 8, on the basis of the observation workflow estimated by the estimating unit 862. Specifically, the setting unit 863 sets transmission image data groups such that the entire image of the selected image data groups can be viewed from a higher perspective among the selected image data groups when the estimating unit 862 estimates the observation workflow as the new observation workflow for performing new observation on the image data groups; sets transmission image data groups with reference to an imaging time of image data that is present immediately before the observation is suspended among the selected image data groups when the estimating unit 862 estimates the observation workflow as the suspended observation resume workflow for suspending observation on the image data groups and thereafter resuming the observation; and sets only image data to which predetermined information is added as transmission image data groups among selected image data groups when the estimating unit 862 estimates the observation workflow as the observation completed examination workflow for performing observation again after observation is completed.

The transmission control unit 864 controls sequential transmission of the transmission image data groups to the terminal device 8 in accordance with the transmission order that is set by the setting unit 863. Specifically, the transmission control unit 864 transmits, to the server 7, a request signal for controlling sequential transmission of the transmission image data groups that are set by the setting unit 863 to the terminal device 8.

Observation Screen Displayed by Terminal Device

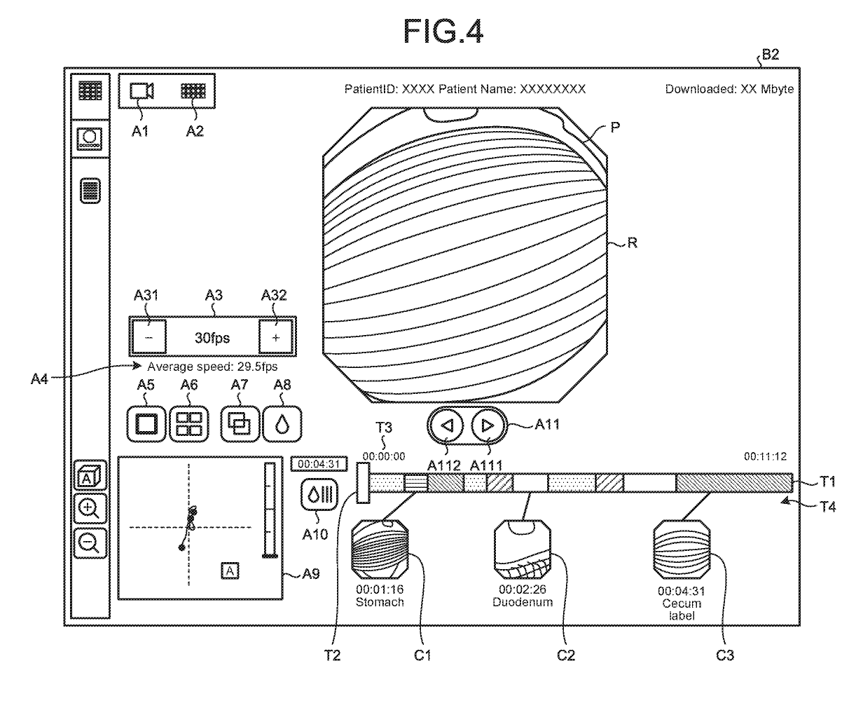

Next, an observation screen that is displayed by the terminal device 8 as described above will be described in detail. FIG. 4 is a diagram illustrating an example of the observation screen that is displayed by the terminal device 8.

As illustrated in FIG. 4, an observation screen B2 includes an image region R for displaying an image P corresponding to image data, a moving image icon A1 for receiving input of an instruction signal for designating a moving image playback mode for sequentially reproducing image data groups in a chronological order, an overview icon A2 for receiving input of an instruction signal for designating an overview mode for displaying a list of only a predetermined number of image data groups, a frame rate icon A3 for receiving input of an instruction signal for setting a playback frame rate for moving images, a display region A4 for displaying an actual playback frame (actual speed), a single display mode icon A5 for receiving input of an instruction signal for designating a single display mode for displaying image data groups one by one, a multiple display mode icon A6 for receiving input of an instruction signal for designating a multiple display mode for simultaneously displaying a plurality of moving image frames, a feature image playback mode icon A7 for receiving input of an instruction signal for designating a feature image playback mode for displaying a feature image that is extracted from the image data groups under a specific condition, a red mode icon A8 for receiving input of an instruction signal for designating a bleeding mode for extracting red image data that includes bleeding or red from the image data groups and displaying a red image, a three-dimensional mode icon A9 for receiving input of an instruction signal for designating a three-dimensional mode for schematically and three-dimensionally displaying a position of the capsule endoscope at the time of capturing an image, a mode display region A10 for displaying a selected playback mode, and a frame designating unit A11 for receiving input of an instruction signal for designating fast forward or fast rewind of frames of image data to be reproduced.

Further, the observation screen B2 includes an average color bar T1 for a selected case, a slide bar T2 for selecting an image corresponding to the average color bar T1, a time of the slide bar T2 (indicating a selected position), a time code T3, a download bar T4 for indicating a download status of the selected case, and captured images C1 to C3 that are selected cases and that are already observed and captured.

Furthermore, the frame rate icon A3 includes a down icon A31 for receiving input of an instruction signal for reducing a frame rate, and an up icon A32 for receiving input of an instruction signal for increasing a frame rate. Moreover, the frame instructing unit A11 includes a fast forward icon A111 for receiving input of an instruction signal for designating fast forward of frames of images, and a fast rewind icon A112 for receiving input of an instruction signal for designating fast rewind of frames of images.

Meanwhile, in the observation screen B2, it may be possible to provide, on the same line as the feature image playback mode icon A7 and the red mode icon A8, a normal mode icon for receiving input of an instruction signal for designating a normal mode for sequentially reproducing all of the image data groups without extracting feature image data and image data containing intensive reddish components from the image data groups, and an auto speed mode icon for receiving input of an instruction signal for designating an auto speed mode (auto speed adjust) for sequentially reproducing only part of image data that is extracted from the image data groups under a predetermined condition. In these two modes, not all of the image data groups of a selected case are displayed in the image region R of the observation screen B2, so that it is possible to reduce the communication traffic of data to be downloaded from the

Processes performed by Terminal Device

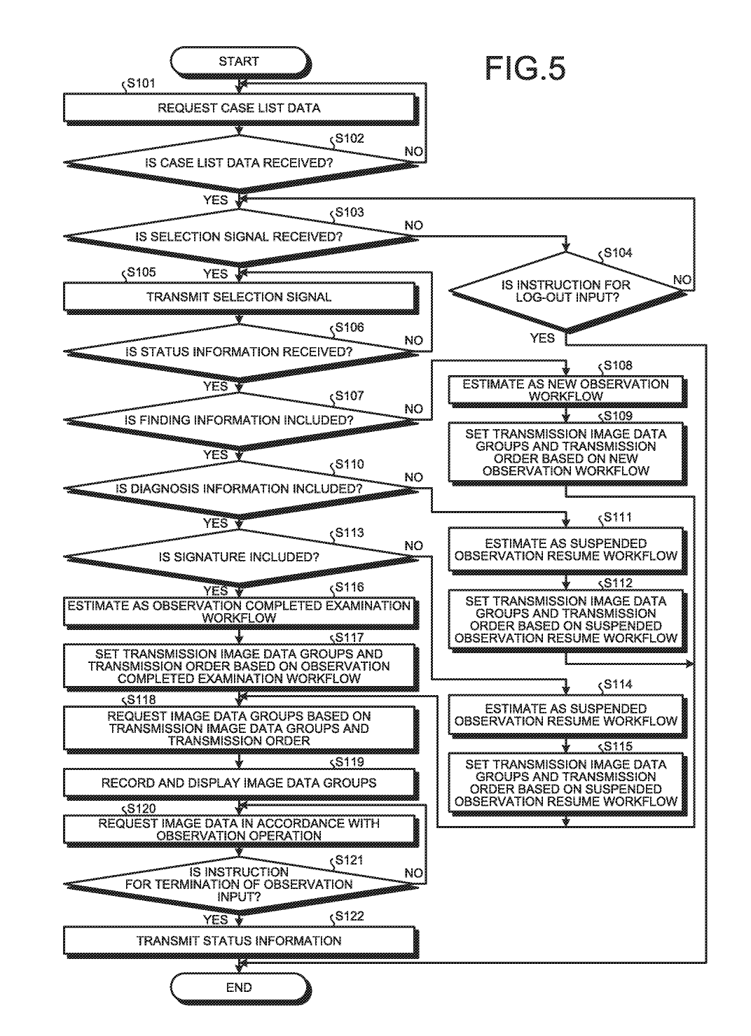

Next, a process performed by the terminal device 8 when a user, such as a doctor, performs observation on a predetermined case file recorded in the server 7 by using the terminal device 8 will be described. FIG. 5 is a flowchart illustrating the outline of the process performed by the terminal device 8.

As illustrated in FIG. 5, first, when the user operates the operation input unit 84 and inputs an instruction signal for designating start of observation, the transmission control unit 864 transmits, to the server 7, a request signal for requesting case list data (a case file in which an examination ID and image data groups are associated) (Step S101).

Subsequently, if the case list data is received from the server 7 (Step S102: Yes), the terminal device 8 proceeds to Step S103 to be described below. In contrast, if the case list data is not received from the server 7 (Step S102: No), the terminal device 8 returns to Step S101 described above.

At Step S103, if a selection signal for selecting any of a plurality of cases displayed by the display unit 82 is input from the operation input unit 84 (Step S103: Yes), the terminal device 8 proceeds to Step S105 to be described later. In contrast, if the selection signal for selecting any of the plurality of cases displayed by the display unit 82 is not input from the operation input unit 84 (Step S103: No), the terminal device 8 proceeds to Step S104 to be described below.

At Step S104, if an instruction signal for log-out is input from the operation input unit 84 (Step S104: Yes), the terminal device 8 terminates the process. In contrast, if the instruction signal for log-out is not input from the operation input unit 84 (Step S104: No), the terminal device 8 returns to Step S103 described above.

At Step S105, the transmission control unit 864 transmits the selection signal input from the operation input unit 84 to the server 7.

Subsequently, if status information on the image data groups of the selected case file is received from the server 7 via the operation input unit 84 (Step S106: Yes), the terminal device 8 proceeds to Step S107 to be described below. In contrast, if the status information on the image data groups of the selected case file is not received from the server 7 via the operation input unit 84 (Step S106: No), the terminal device 8 returns to Step S105 described above.

At Step S107, the estimating unit 862 determines whether finding information on findings of the user is included in the status information on the image data groups of the case file. If the estimating unit 862 determines that the finding information on findings of the user is included in the status information on the image data groups of the case file (Step S107: Yes), the terminal device 8 proceeds to Step S110 to be described later. In contrast, if the estimating unit 862 determines that the finding information on findings of the user is not included in the status information on the image data groups of the case file (Step S107: No), the terminal device 8 proceeds to Step S108 to be described below.

At Step S108, because the finding information on findings of the user is not included in the status information on the image data groups of the case file, the estimating unit 862 estimates an observation workflow for the image data groups of the case file as the new observation workflow for performing new observation on the image data groups. Here, the new observation workflow is any of the followings: the user performs observation on the image data groups of the case file for the first time; the user has once checked only outline of the image data groups of the case file and performs observation in detail for the first time; and the user has completed observation on the image data groups of the case file but a different user performs observation again from the beginning.

Subsequently, the setting unit 863 sets transmission image data groups and a transmission order of optimally browsing images on the terminal device 8 among the image data groups of the case file selected by the operation input unit 84, on the basis of the new observation workflow estimated by the estimating unit 862 (Step S109). After Step S109, the terminal device 8 proceeds to Step S118 to be described later. Specifically, the setting unit 863 sets transmission image data groups and a transmission order such that the entire image of the image data groups can be viewed from a higher perspective among the image data groups of the case file. Meanwhile, the setting unit 863 may set the transmission order of the image data groups of the case file such that transmission is performed from the top image data (with the oldest imaging time) of the image data groups, or may set the transmission order such that transmission is performed from the first image data in which a predetermined organ, such as a small intestine (image of duodenum in which pylorus appears) appears. Further, when setting the transmission image data groups and the transmission order such that the entire image of the image data groups can be viewed from a higher perspective, the setting unit 863 sets, as transmission image data, an image data group corresponding to the average color bar T1, an image data group including a first predetermined number of images (for example, 100 images) among chronological images, and an image data group in which red color is detected, among the image data groups of the case file. Then, the setting unit 863 sets the transmission order in order of, for example, the image data group corresponding to the average color bar T1, the image data group including the first predetermined number of images (for example, 100 images) among the chronological images, and the image data group in which red color is detected. In this case, when the user selects an image using the slide bar T2 (see FIG. 4), the setting unit 863 sets a predetermined number of images (for example, 100 images) after the time of the image that is selected by the user using the slide bar T2 (see FIG. 4) and a predetermined number of images (for example, 100 images) before the time of the image that is selected by the user using the slide bar T2, as the transmission image data groups. Further, the setting unit 863 sets the transmission order in order of the predetermined number of images after the time of the image that is selected by the user using the slide bar T2 and the predetermined number of images before the time of the image that is selected by the user using the slide bar T2.

Furthermore, when the user selects the overview mode for checking the outline of the image data groups of the selected case file, the setting unit 863 sets the image data group corresponding to the average color bar T1 and the image data group for the overview screen as the transmission image data groups among the image data groups of the case file, and sets the transmission order in order of the image data group corresponding to the average color bar T1 and the image data group for the overview screen. In this case, when the user selects an image in the overview screen displayed by the display unit 82, the setting unit 863 further sets the transmission order in order of a predetermined number of images (for example, 20 images) after the time of the selected image and a predetermined number of images (for example, 20 images) before the time of the selected image.

At Step S110, the estimating unit 862 determines whether diagnosis information is included in the status information of the image data groups of the case file. If the estimating unit 862 determines that the diagnosis information is included in the status information of the image data groups of the case file (Step S110: Yes), the terminal device 8 proceeds to Step S113 to be described later. In contrast, if the estimating unit 862 determines that the diagnosis information is not included in the status information of the image data groups of the case file (Step S110: No), the terminal device 8 proceeds to Step S111 to be described below.

At Step S111, because the finding information is included in the status information of the image data groups of the case file but the diagnosis information is not included, the estimating unit 862 estimates an observation workflow for the image data groups as the suspended observation resume workflow for suspending observation on the image data groups of the case file and thereafter resuming the observation. Here, the suspended observation resume workflow means that observation has been performed halfway with respect to the image data groups of the case file and the same person continues the observation at a different place or a different time.

Thereafter, the setting unit 863 sets transmission image data groups and a transmission order of optimally browsing images on the terminal device 8 among the image data groups of the case file selected by the operation input unit 84, on the basis of the suspended observation resume workflow estimated by the estimating unit 862 (Step S112). After Step S112, the terminal device 8 proceeds to Step S118 to be described later. Specifically, if a predetermined time (for example, 24 hours) has not elapsed since previous observation, the setting unit 863 sets image data groups including a predetermined number of images before and after a predetermined time with reference to an imaging time of an image that is present immediately before the observation on the image data groups of the case file is suspended. More specifically, the setting unit 863 sets a predetermined number of images (for example, 100 images) after an imaging time of an image corresponding to the current slide bar T2 and a predetermined number of images (for example, 100 images) before the imaging time of the image corresponding to the current slide bar T2 as the transmission image data groups. Furthermore, the setting unit 863 sets the transmission order in order of the predetermined number of images (for example, 100 images) after the imaging time of the image corresponding to the current slide bar T2 and the predetermined number of images (for example, 100 images) before the imaging time of the image corresponding to the current slide bar T2.

Moreover, when a predetermined time (for example, 24 hours) has elapsed since previous observation and the outline of the image data groups of the case file is to be checked, the setting unit 863 sets, as the transmission image data groups, an image data group corresponding to the average color bar T1, an image data group including a predetermined number of images (for example, 100 images) after the imaging time of the image corresponding to the current slide bar T2, and an image data group including a predetermined number of images (for example, 100 images) before the imaging time of the image corresponding to the current slide bar T2. Furthermore, the setting unit 863 sets the transmission order in order of the image data group corresponding to the average color bar, the image data group including the predetermined number of images (for example, 100 images) after the imaging time of the image corresponding to the current slide bar T2, and the image data group including the predetermined number of images (for example, 100 images) before the imaging time of the image corresponding to the current slide bar T2.

At Step S113, the estimating unit 862 determines whether a signature (sign) indicating completion of a diagnosis by the user who has written the diagnosis information is included in the status information on the image data groups of the case file. If the estimating unit 862 determines that the signature of the user with respect to the diagnosis information is included in the status information on the image data groups of the case file (Step S113: Yes), the terminal device 8 proceeds to Step S116 to be described later. In contrast, if the estimating unit 862 determines that the signature of the user with respect to the diagnosis information is not included in the status information on the image data groups of the case file (Step S113: No), the terminal device 8 proceeds to Step S114 to be described below.

At Step S114, because the finding information and the diagnosis information are included in the status information on the image data groups of the case file but the signature indicating completion of the observation by the user with respect to the diagnosis information is not included, the estimating unit 862 estimates an observation workflow for the image data groups of the case file as the suspended observation resume workflow.

Thereafter, the setting unit 863 sets transmission image data groups and a transmission order of optimally browsing images on the terminal device 8 among the image data groups of the case file selected by the operation input unit 84, on the basis of the suspended observation resume workflow estimated by the estimating unit 862 (Step S115). After Step S115, the terminal device 8 proceeds to Step S118 to be described later. Specifically, if a predetermined time (for example, 24 hours) has not elapsed since previous observation, the setting unit 863 sets an image data group corresponding to the average color bar T1 and image data groups including a predetermined number of images before and after a predetermined time with reference to an imaging time of an image that is present immediately before the observation on the image data groups of the case file is suspended. More specifically, the setting unit 863 sets the image data group corresponding to the average color bar T1 as the transmission image data group, and further sets the image data group including the predetermined number of images (for example, 100 images) after the imaging time of the image corresponding to the current slide bar T2 and the image data group including the predetermined number of images (for example, 100 images) before the imaging time of the image corresponding to the current slide bar T2 as the transmission image data groups. Furthermore, the setting unit 863 sets the transmission order in order of the image data group corresponding to the average color bar T1, the image data group including the predetermined number of images (for example, 100 images) after the imaging time of the image corresponding to the current slide bar T2, and the image data group including the predetermined number of images (for example, 100 images) before the imaging time of the image corresponding to the current slide bar T2.

At Step S116, because the finding information and the diagnosis information are included in the status information on the image data groups of the case file and the signature indicating completion of the diagnosis by the user who has written the diagnosis information is also included, the estimating unit 862 estimates an observation workflow for the image data groups of the case file as the observation completed examination workflow. Here, the observation completed examination workflow is any of the followings: after the user performs observation once on the image data groups of the case file, the user performs observation again because there is a period or site of interest; the user has completed observation on the image data groups of the case file, but a different user performs final observation while confirming contents of the observation result; the user has completed observation on the image data groups of the case file, but observation is performed as a reference when observation on an image data group of a different subject (patient) or observation on image data groups of a different case file is performed; the user has completed observation on the image data groups of the case file, but observation is performed with reference to a past result because an examination on the same subject (same patient) as the image data groups of the case file is performed again (follow-up examination or re -examination); and the user has completed observation on the image data groups of the case file, but observation is performed for the purpose of demonstration for other people because the case is a pathognomonic case.

Subsequently, the setting unit 863 sets transmission image data groups and a transmission order of optimally browsing images on the terminal device 8 among the image data groups of the case file selected by the operation input unit 84, on the basis of the observation completed examination workflow estimated by the estimating unit 862 (Step S117). After Step S117, the terminal device 8 proceeds to Step S118 to be described later. Specifically, if a predetermined time has elapsed since previous observation and a picked-up image is to be checked, the setting unit 863 sets, as the transmission image data groups, an image data group including a predetermined number of images (for example, 30 images) after an imaging time of the picked-up image (captured image), an image data group including a predetermined number of images (for example, 30 images) before the imaging time of the picked -up image (captured image), an image data group including a predetermined number of images (for example, 100 images) after an imaging time of an image corresponding to the current slide bar T2, and an image data group including a predetermined number of images (for example, 100 images) before the imaging time of the image corresponding to the current slide bar T2. Furthermore, if a predetermined time has elapsed since previous observation and the picked-up image is to be checked, the setting unit 863 sets the transmission order in order of the predetermined number of images (for example, 30 images) after the imaging time of the picked-up image (captured image), the predetermined number of images (for example, 30 images) before the imaging time of the picked-up image (captured image), the predetermined number of images (for example, 100 images) after the imaging time of the image corresponding to the current slide bar T2, and the predetermined number of images (for example, 100 images) before the imaging time of the image corresponding to the current slide bar T2.

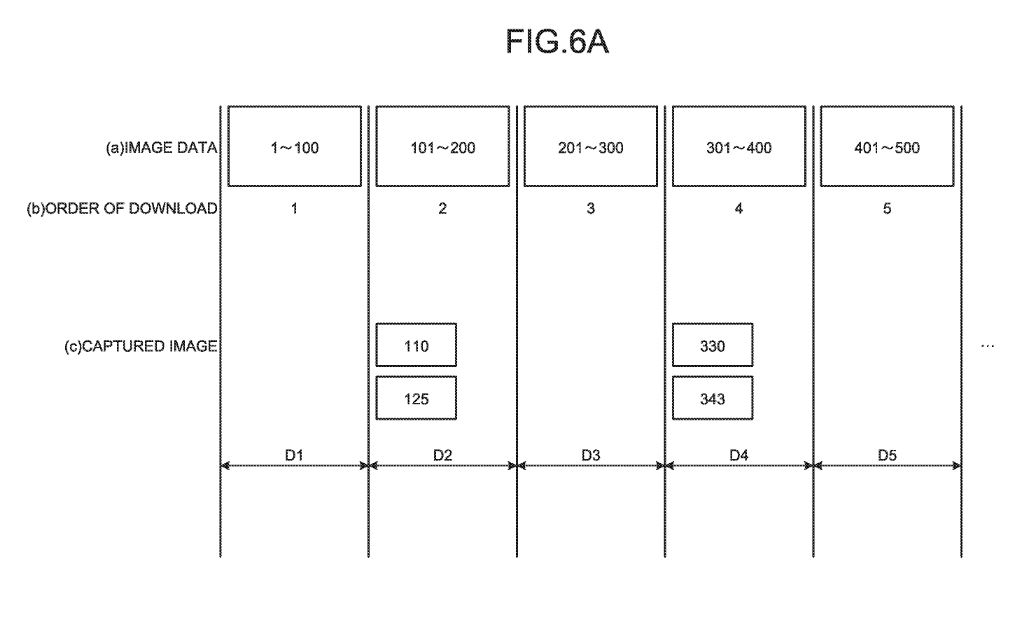

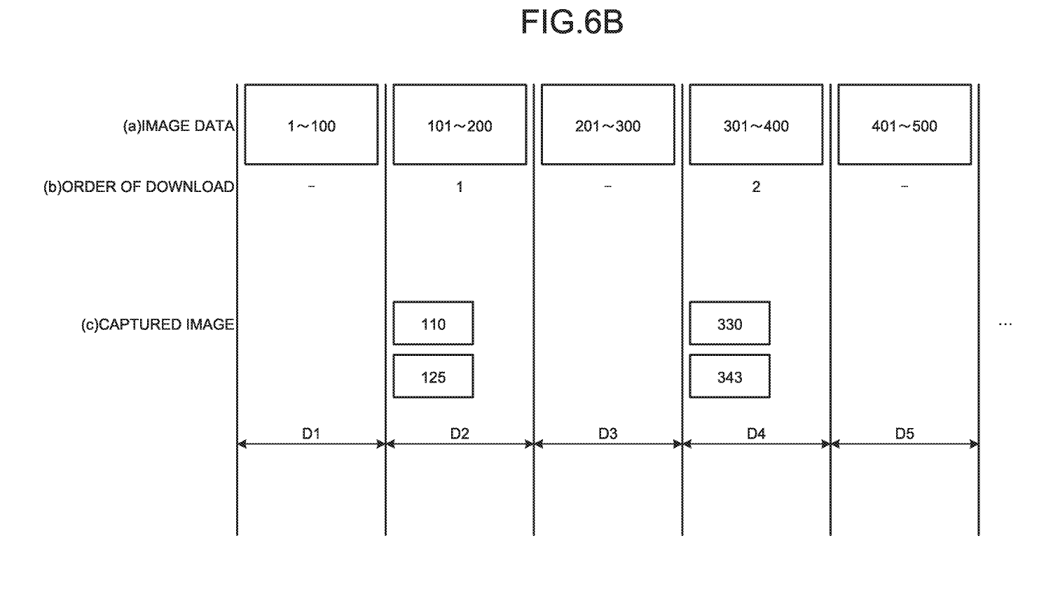

FIG. 6A is a diagram schematically illustrating a transmission order of image data groups of a case file that is transmitted to the terminal device 8 in the new observation workflow and captured images. FIG. 6B is a diagram schematically illustrating image data groups of a case file that is transmitted to the terminal device 8 in the observation completed examination workflow, a transmission order of the image data groups, and images captured in the new observation workflow. Meanwhile, in FIG. 6A and FIG. 6B, numbers indicate an imaging order of images in the image data groups.

As illustrated in FIG. 6A, in the case of the new observation workflow, the terminal device 8 normally requests the image data groups in the imaging order with respect to the image data groups of the case file. At this time, the user picks up a feature image via the operation input unit 84 while viewing images that are displayed on the display unit 82 in the new observation workflow. For example, the user picks up an image 110 and an image 125 as feature images in a period D2 (an image 101 to an image 200) of the image data group. Further, the user picks up an image 330 and an image 343 as feature images in a period D4 of the image data group (an image 301 to an image 400). Furthermore, the observation operation is not always performed only once but the same user may perform observation a plurality of number of times or a different user may perform observation. Moreover, a different hospital or an inspection agency may be commissioned to perform observation operation and matching may be performed. Even when the observation operation is performed a plurality of number of times, it is highly likely that the same image is picked up as a feature image. Therefore, in the observation completed examination workflow (the second or later observation workflow on the same case file), the setting unit 863 sets images near a captured image that has been picked up as a feature image in the previous observation operation as the transmission image data groups, and sets the transmission order among the transmission image data groups near the captured image.

Specifically, as illustrated in FIG. 6B, when the estimating unit 862 estimates the workflow as the observation completed examination workflow, the setting unit 863 sets only image data (for example, the image 101 to the image 200 in the period D2 and the image 301 to the image 400 in the period D4) near the captured images that has been picked up as feature images in the previous observation operation as the transmission image data groups. More specifically, the setting unit 863 sets, as the transmission image data groups, a predetermined number of images (for example, 30 images) after the imaging time of the picked-up image (captured image), a predetermined number of images (for example, 30 images) before the imaging time of the picked-up image (captured image), and the image data group corresponding to the average color bar, among the image data groups of the case file. Further, the setting unit 863 sets the transmission order in order of the predetermined number of images (for example, 30 images) after the imaging time of the picked-up image (captured image), the predetermined number of images (for example, 30 images) before the imaging time of the picked-up image (captured image), and the image data group corresponding to the average color bar. With this configuration, it is possible to reduce a download wait time during observation. Moreover, the images near the captured image that has been picked-up as the feature image are set as the transmission image data groups, and unnecessary image data is not downloaded, so that it is possible to reduce the communication traffic. As a result, it is possible to reduce a communication cost.

At Step S118, the transmission control unit 864 transmits a request signal for requesting image data groups from the server 7, on the basis of the transmission image data groups and the transmission order set by the setting unit 863.

Subsequently, the transmission control unit 864 receives image data from the server 7, records the image data in the temporary recording unit 853, and displays the image data on the display unit 82 (Step S119).

Thereafter, the transmission control unit 864 transmits a request signal for requesting image data from the server 7, in accordance with observation operation that is performed by the user via the operation input unit 84 (Step S120).

Subsequently, if a termination signal for designating termination of observation is input from the operation input unit 84 (Step S121: Yes), the transmission control unit 864 associates operation status information related to an operation history of the observation on the case file with the status information on the case file, and transmits the status information to the server 7 (Step S122). After Step S122, the terminal device 8 terminates the process.

At Step S121, if the termination signal for designating termination of observation is not input from the operation input unit 84 (Step S121: No), the terminal device 8 returns to Step S120 described above. According to the first embodiment described above, the estimating unit 862 estimates a type of the observation workflow that is applied when browsing image data groups on the terminal device 8, on the basis of the status information on the image data groups; the setting unit 863 sets the transmission image data groups to be transmitted to the terminal device 8 among the selected image data groups on the basis of the observation workflow estimated by the estimating unit 862; and the transmission control unit 864 controls transmission of the transmission image data groups set by the setting unit 863 to the terminal device 8. Therefore, it is possible to effectively transmit only requisite minimum image data groups depending on the type of the observation workflow. As a result, it is possible to effectively download image data, so that it is possible to minimize a wait time of the user.

Further, according to the first embodiment, when the estimating unit 862 estimates the observation workflow as the observation completed examination workflow, the setting unit 863 sets only image data to which predetermined information is added in the image data groups as the transmission image data groups, so that it is possible to prevent transmission of unnecessary image data. As a result, it is possible to reduce the communication traffic, so that it is possible to prevent an increase in a communication cost.

### Modification of First Embodiment

Next, a modification of the first embodiment will be described. In a first modification of the first embodiment, a method of setting the transmission image data groups based on the observation completed examination workflow that is set by the setting unit of the terminal device is different. In the following, the method of setting the transmission image data groups based on the observation completed examination workflow that is set by a setting unit of a terminal device according to the modification of the first embodiment will be described. The same components as those of the endoscope system 1 according to the first embodiment as described above are denoted by the same reference signs, and explanation thereof will be omitted.

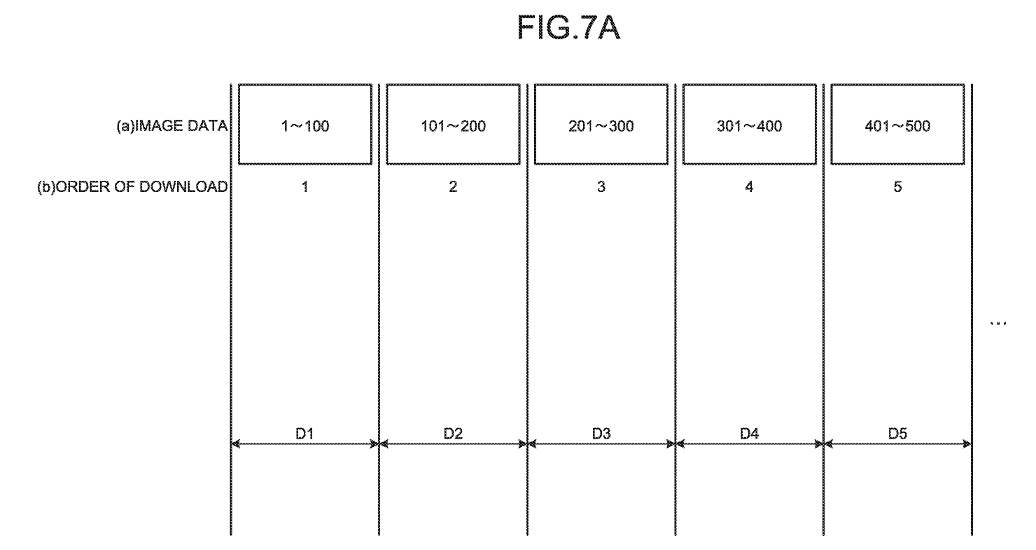

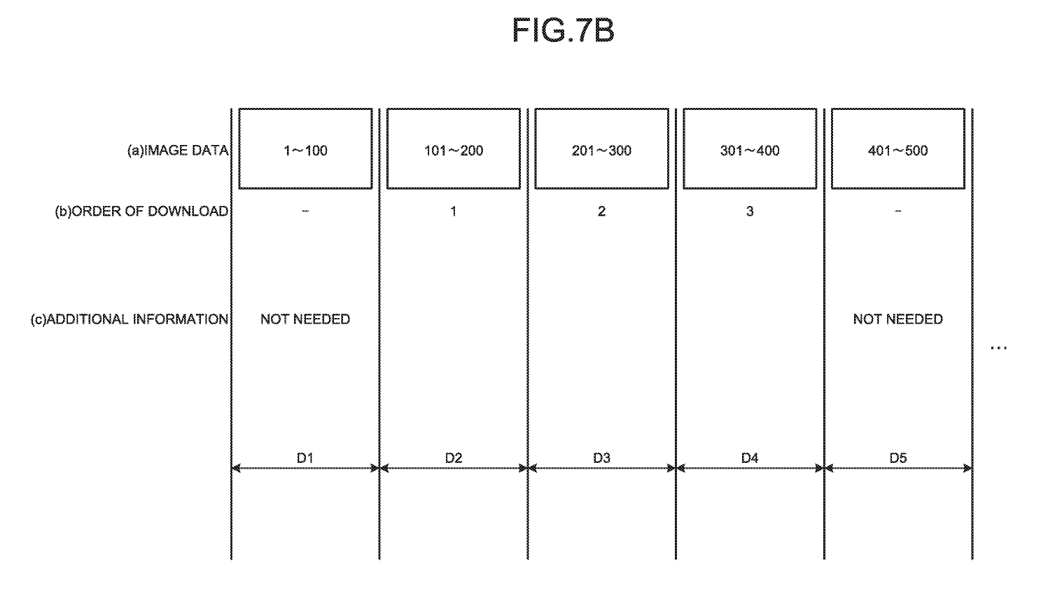

FIG. 7A is a diagram schematically illustrating a transmission order of image data groups of a case file that is transmitted to the terminal device 8 in the new observation workflow. FIG. 7B is a diagram schematically illustrating image data groups of a case file that is transmitted to the terminal device 8 in the observation completed examination workflow and a transmission order. Meanwhile, in FIG. 7A and FIG. 7B, numbers indicate an imaging order of images in the image data groups.

As illustrated in FIG. 7A, in the case of the new observation workflow, the terminal device 8 requests the image data groups in the imaging order with respect to the image data groups of the case file. At this time, the user excludes images that are not needed for observation via the operation input unit 84 while viewing images that are displayed on the display unit 82 in the new observation workflow. For example, the user excludes, as unnecessary images, image data groups corresponding to a period D1 and a period D5 among the image data groups. Here, the unnecessary images are in-vitro images, images capable of identifying personal information, images in which a regional site appears and which may invade privacy, images of regions other than a target (for example, in the case of a capsule endoscope for a small intestine, an esophagus, a stomach, and a large intestine), images with low image quality and inappropriate for observation, and images in which a feature region does not appear. Therefore, in the observation completed examination workflow (the second or later observation workflow on the same case file), the setting unit 863 excludes, from the transmission image data groups, image data groups that have been excluded as images that are not needed for observation in the previous observation operation.

Specifically, as illustrated in FIG. 7B, when the estimating unit 862 estimates the workflow as the observation completed examination workflow, the setting unit 863 sets the transmission image data groups while excluding the period D1 (an image 1 to an image 100) and the period D5 (an image 401 to an image 500) that are determined as being not needed for observation in the previous observation operation. With this configuration, it is possible to reduce a download wait time at the time of observation. Further, the transmission image data groups are set while excluding the image data groups that are determined as being not needed for observation, so that it is possible to prevent download of unnecessary image data. As a result, it is possible to reduce the communication traffic, so that it is possible to prevent an increase in a communication cost.

According to the modification of the first embodiment described above, when the estimating unit 862 estimates the workflow as the observation completed examination workflow, the setting unit 863 sets the transmission image data groups while excluding the period D1 (the image 1 to the image 100) and the period D1 (the image 401 to the image 500) that are determined as being not needed for observation in the previous observation operation; therefore, it is possible to reduce a download wait time at the time of observation, and it is possible to set the transmission image data groups while excluding the image data groups that are determined as being not needed for observation, so that it is possible to prevent download of unnecessary image data. As a result, it is possible to reduce the communication traffic, so that it is possible to prevent an increase in a communication cost.

### Second Embodiment

Next, a second embodiment will be described. An endoscope system according to the second embodiment has the same configuration as the endoscope system 1 according to the first embodiment as described above, but a terminal device performs a different process. In the following, a process performed by a terminal device according to the second embodiment will be described. The same components as those of the endoscope system 1 according to the first embodiment as described above are denoted by the same reference signs, and explanation thereof will be omitted.

Processes performed by Terminal Device

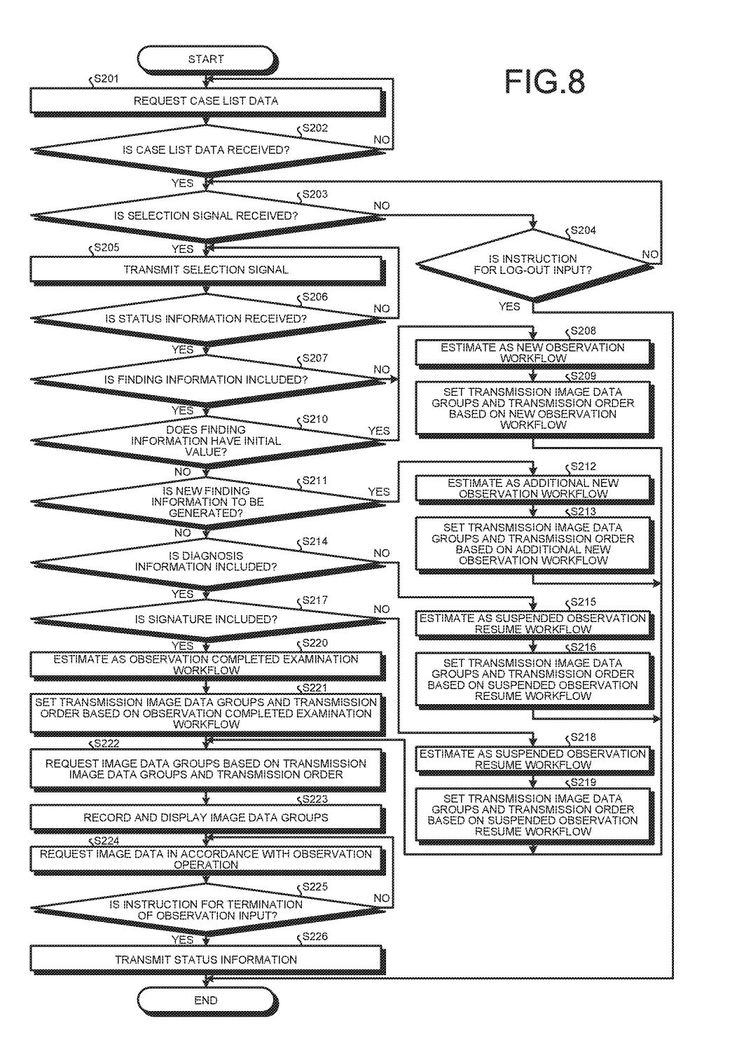

FIG. 8 is a flowchart illustrating the outline of a process performed by the terminal device 8 according to the second embodiment. In FIG. 8, Step S201 to Step S209 respectively correspond to Step S101 to Step S109 in FIG. 5 as described above.

At Step S210, the estimating unit 862 determines whether the finding information has an initial value (Default). Specifically, if the estimating unit 862 determines that the finding information has the initial value (Step S210: Yes), the terminal device 8 proceeds to Step S208. In contrast, if the estimating unit 862 determines that the finding information does not have the initial value (Step S210: No), the terminal device 8 proceeds to Step S211 to be described below.

At Step S211, the estimating unit 862 determines whether to generate new finding information on the image data groups of the case file, on the basis of the status information on the image data groups of the case file. Specifically, the estimating unit 862 determines whether an instruction signal for designating generation of new finding information is input from the operation input unit 84 although one or more pieces of finding information are already present, on the basis of the status information on the image data groups of the case file. If the estimating unit 862 determines that new finding information with respect to the image data groups of the case file is to be generated (Step S211: Yes), the terminal device 8 proceeds to Step S212 to be described later. In contrast, if the estimating unit 862 determines that new finding information with respect to the image data groups of the case file is not to be generated (Step S211: No), the terminal device 8 proceeds to Step S214 to be described later.

At Step S212, because the finding information is included in the status information on the image data groups of the case file and the instruction signal for designating generation of new finding information is input from the operation input unit 84 although the finding information does not have the initial value, the estimating unit 862 estimates an observation workflow for the image data groups of the case file as an additional new observation workflow.

Subsequently, the setting unit 863 sets transmission image data groups and a transmission order of optimally browsing images on the terminal device 8 among the image data groups of the case file selected by the operation input unit 84, on the basis of the additional new observation workflow estimated by the estimating unit 862 (Step S213). In this case, as in the modification of the first embodiment as described above, the setting unit 863 sets the transmission image data groups while excluding image data groups that are determined as being not needed for observation among the image data groups of the case file. With this configuration, even when the finding information is included in the status information on the image data groups of the case file and new finding information is to be generated although the finding information does not have the initial value, it is possible to prevent download of unnecessary image data, so that it is possible to reduce the communication traffic and reduce a wait time due to unnecessary download. After Step S213, the terminal device 8 proceeds to Step S222 to be described below.

Step S214 to Step S226 respectively correspond to Step S110 to Step S122 in FIG. 5. After Step S226, the terminal device 8 terminates the process.

According to the second embodiment described above, it is possible to effectively transmit only requisite minimum image data groups depending on the type of the observation workflow. As a result, it is possible to effectively download image data, so that it is possible to minimize a wait time of the user.

Further, according to the second embodiment, the setting unit 863 sets only transmission image data groups while excluding the image data groups that are determined as being not needed for observation from the transmission image data groups, so that it is possible to prevent transmission of unnecessary image data. As a result, it is possible to reduce the communication traffic, so that it is possible to prevent an increase in a communication cost.

### Third Embodiment

Next, a third embodiment will be described. An endoscope system according to the third embodiment has the same configuration as the endoscope system 1 according to the first embodiment as described above, but a terminal device performs a different process. Specifically, in the first embodiment as described above, the transmission image data groups in the image data groups are set by estimating a type of the observation workflow corresponding to a status of the image data groups on the basis of the finding information, the diagnosis information, and the signature that are included in the status information on the image data groups of the case file; however, in the third embodiment, transmission image data groups in the image data groups of the case file are set by estimating a type of the observation workflow on the basis of log information that is included in the status information on the image data groups of the case file. In the following, a process performed by a terminal device according to the third embodiment will be described. The same components as those of the endoscope system 1 according to the first embodiment as described above are denoted by the same reference signs, and explanation thereof will be omitted.

Processes performed by Terminal Device

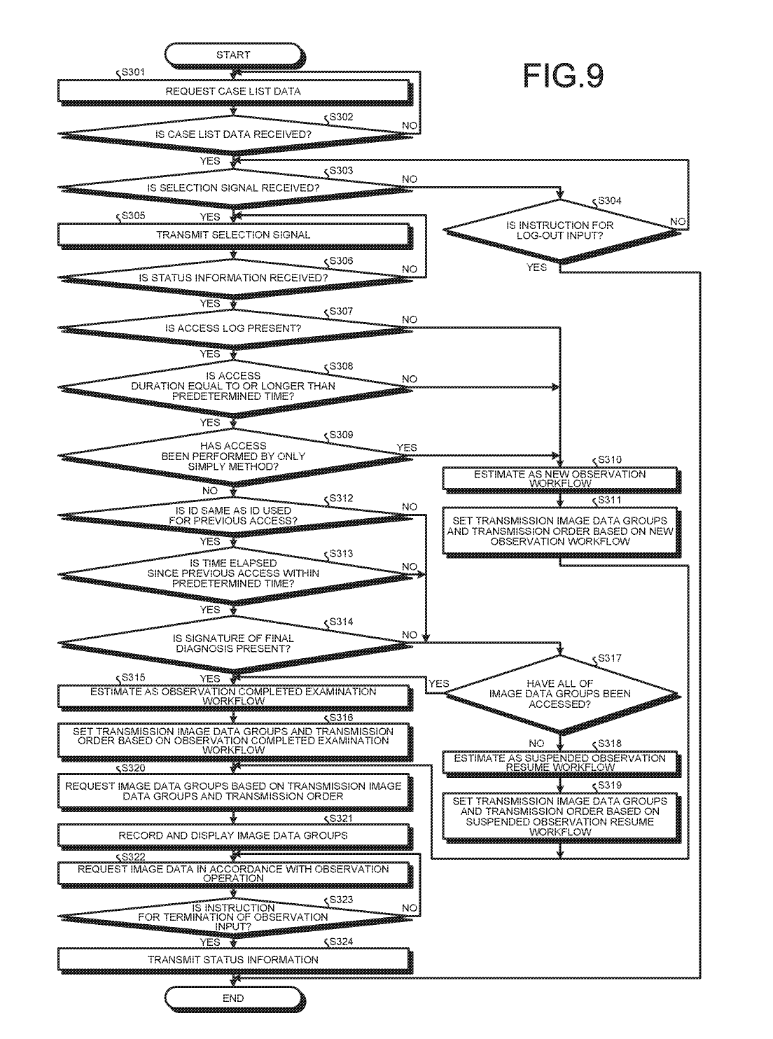

FIG. 9 is a flowchart illustrating the outline of a process performed by the terminal device 8 according to the third embodiment. In FIG. 9, Step S301 to Step S306 respectively correspond to Step S101 to Step S106 in FIG. 5 as described above.

At Step S307, the estimating unit 862 determines whether an access log of the terminal device 8 with respect to the image data groups of the case file is present, on the basis of log information that is included in the status information on the image data groups of the case file selected via the operation input unit 84. Here, the log information is access log information on access to the image data groups by the terminal device 8, operation log information at the time of browsing image data by the terminal device 8, browsing mode log information on a browsing mode at the time of browsing the image data groups by the terminal device 8, identification log information for identifying a user who has performed observation on the image data groups, and browsing time log information on a browsing time at the time of browsing the image data groups by the terminal device 8. If the estimating unit 862 determines that the access log of the terminal device 8 with respect to the image data groups of the case file is present (Step S307: Yes), the terminal device 8 proceeds to Step S308 to be described later. In contrast, if the estimating unit 862 determines that the access log of the terminal device 8 with respect to the image data groups of the case file is not present (Step S307: No), the terminal device 8 proceeds to Step S310 to be described later.

At Step S308, the estimating unit 862 determines whether the duration of access to the image data groups of the case file by the terminal device 8 is equal to or longer than a predetermined time, on the basis of the log information included in the status information on the image data groups of the case file. Specifically, the estimating unit 862 determines whether the duration of access to the image data groups of the case file by the terminal device 8 is equal to or longer than a predetermined time, such as 10 minutes, on the basis of the browsing time log information of the log information included in the status information of the case file. If the estimating unit 862 determines that the duration of access to the image data groups of the case file by the terminal device 8 is equal to or longer than the predetermined time (Step S308: Yes), the terminal device 8 proceeds to Step S309 to be described later. In contrast, if the estimating unit 862 determines that the duration of access to the image data groups of the case file by the terminal device 8 is not equal to or longer than the predetermined time (Step S308: No), the terminal device 8 proceeds to Step S310 to be described later.

At Step S309, the estimating unit 862 determines whether the terminal device 8 has accessed the image data groups of the case file by only a simple method, on the basis of the log information included in the status information on the image data groups of the case file. Specifically, the estimating unit 862 determines whether the terminal device 8 has performed access by only a simple method by determining whether the terminal device 8 has observed the entire image data groups of the case file, whether a frame rate that is set at the time of the observation is fast (for example, 240 fps), or whether the observation is performed on the image data groups of the case file using a list mode, such as an overview mode, on the basis of the browsing mode log information or the browsing time log information of the log information included in the status information on the image data groups of the case file. If the estimating unit 862 determines that the terminal device 8 has accessed the image data groups of the case file by only a simple method (Step S309: Yes), the terminal device 8 proceeds to Step S310 to be described later. In contrast, if the estimating unit 862 determines that the terminal device 8 has not accessed the image data groups of the case file by only a simple method (Step S309: No), the terminal device 8 proceeds to Step S312 to be described later. Step S310 and Step S311 respectively correspond to

Step S108 and Step S109 in FIG. 5 as described above. After Step S311, the terminal device 8 proceeds to Step S320 to be described later.

At Step S312, the estimating unit 862 determines whether the terminal device 8 has the same ID as the terminal device 8 that has previously accessed the image data groups of the case file, on the basis of the log information included in the status information on the image data groups of the case file. Specifically, the estimating unit 862 determines whether the access is performed by a person different from a person who has previously accessed the image data groups of the case file, on the basis of the identification log information of the log information included in the status information on the image data groups of the case file. If the estimating unit 862 determines that the terminal device 8 has the same ID as the terminal device 8 that has previously accessed the image data of the case file (Step S312: Yes), the terminal device 8 proceeds to Step S313 to be described later. In contrast, if the estimating unit 862 determines that the terminal device 8 does not have the same ID as the terminal device 8 that has previously accessed the image data groups of the case file (Step S312: No), the terminal device 8 proceeds to Step S317 to be described later.

At Step S313, the estimating unit 862 determines whether a time elapsed since the time at which the terminal device 8 has previously accessed the case file is within a predetermined time, on the basis of the log information included in the status information on the image data groups of the case file. Specifically, the estimating unit 862 determines whether the time elapsed since the time at which the terminal device 8 has previously accessed the image data groups of the case file is within two weeks, on the basis of the browsing time log information of the log information included in the status information on the image data groups of the case file. If the estimating unit 862 determines that the time elapsed since the time at which the terminal device 8 has previously accessed the image data groups of the case file is within the predetermined time (Step S313: Yes), the terminal device 8 proceeds to

Step S314 to be described later. In contrast, if the estimating unit 862 determines that the time elapsed since the time at which the terminal device 8 has previously accessed the image data groups of the case file is not within the predetermined time (Step S313: No), the terminal device 8 proceeds to Step S317 to be described later.

At Step S314, the estimating unit 862 determines whether a signature of a final diagnosis by the user with respect to the image data groups of the case file is present, on the basis of the log information included in the status information on the image data groups of the case file. If the estimating unit 862 determines that the signature of the final diagnosis by the user with respect to the image data groups of the case file is present (Step S314: Yes), the terminal device 8 proceeds to Step S315 to be described later. In contrast, if the estimating unit 862 determines that the signature of the final diagnosis by the user with respect to the image data groups of the case file is absent (Step S314: No), the terminal device 8 proceeds to Step S317 to be described later.

Step S315 corresponds to Step S116 in FIG. 5 as described above. After Step S315, the terminal device 8 proceeds to Step S316 to be described below.

At Step S316, the setting unit 863 sets transmission image data groups and a transmission order of optimally browsing images on the terminal device 8 with respect to the image data groups of the case file selected by the operation input unit 84, on the basis of the observation completed examination workflow estimated by the estimating unit 862. After Step S316, the terminal device 8 proceeds to Step S320 to be described later.

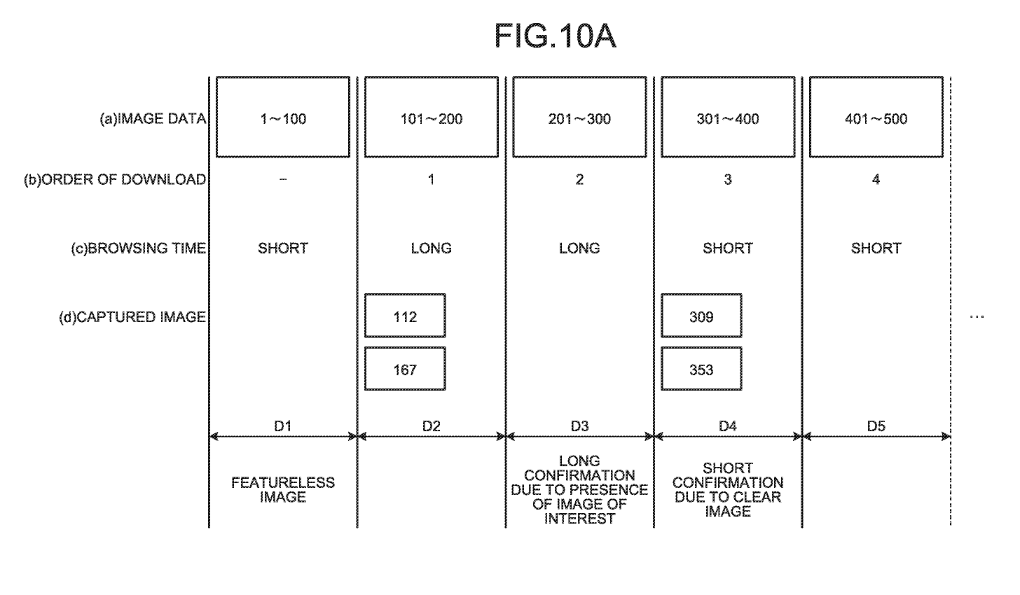

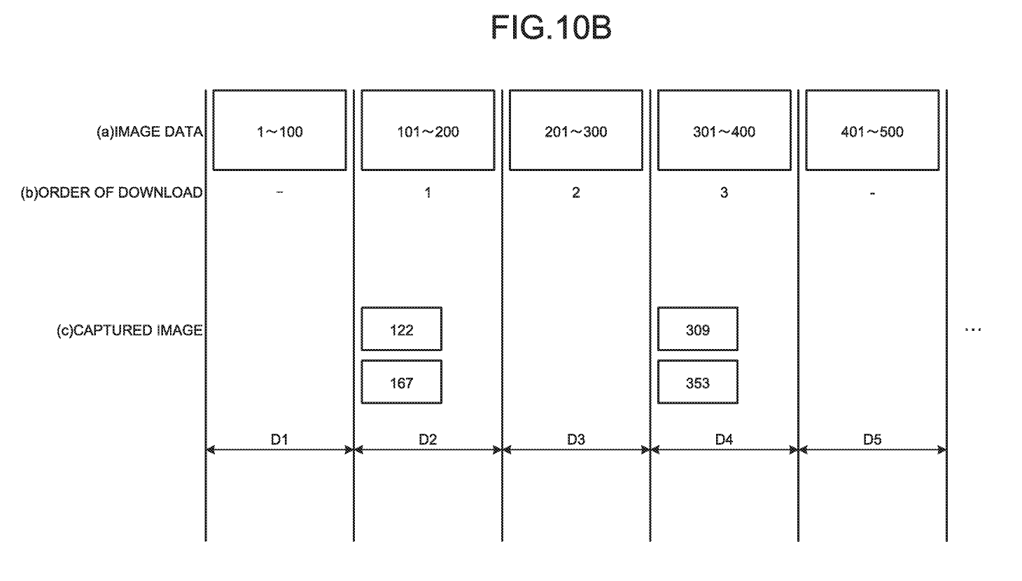

FIG. 10A is a diagram schematically illustrating a transmission order of image data groups of a case file that is transmitted to the terminal device 8 in the new observation workflow, images that are captured in the new observation workflow, and a browsing time for each predetermined period. FIG. 10B is a diagram schematically illustrating image data groups of a case file that is transmitted to the terminal device 8 in the observation completed examination workflow, a transmission order of the image data groups, and images that are captured in the new observation workflow. Meanwhile, in FIG. 10A and FIG. 10B, numbers indicate an imaging order of images in the image data groups. As illustrated in FIG. 10A, in the case of the new observation workflow, the terminal device 8 normally requests the image data groups in the imaging order with respect to the image data groups of the case file. At this time, the user picks up a feature image via the operation input unit 84 while viewing images that are displayed on the display unit 82 in the new observation workflow. For example, the user picks up an image 112 and an image 167 as feature images in the period D2 (the images 101 to 200) of the image data group. Further, the user becomes inclined to re-check an image, which has been checked many times but has not eventually been picked up in the period D2, after the observation during the period D2 (the images 101 to 200) of the image data group. Therefore, in the observation completed examination workflow (the second or later observation workflow on the same case file), the setting unit 863 sets an image data group corresponding to a period in which the observation has been performed for a long time, as a transmission image data group that is to be preferentially downloaded, on the basis of an operation log of the previous observation operation.

Specifically, as illustrated in FIG. 10B, when the estimating unit 862 estimates the workflow as the observation completed examination workflow, the setting unit 863 sets an image data group corresponding to a period in which observation has been performed for a long time in the previous observation operation, as a transmission image data group that is to be preferentially downloaded as compared to the image data groups corresponding to the other periods, on the basis of the operation log of the log information included in the status information on the image data groups of the case file. More specifically, the setting unit 863 sets a transmission order in order of the image data group (the image 101 to the image 200) corresponding to the period D2, the image data group (an image 201 to an image 300) corresponding to a period D3, and the image data group (the image 301 to the image 400) corresponding to the period D4, among the image data groups of the case file. In this case, even when picked-up images (an image 309 and an image 353) are present in the period D4, the setting unit 863 preferentially sets an earlier transmission order of the image data group corresponding to the period D3 in which the observation has been performed for a long time, as compared to the image data group corresponding to the period D4 in which the observation has been performed within a short time. In other words, the setting unit 863 preferentially sets the image data group corresponding to the period in which an observation time is long as the transmission image data group, on the basis of the operation log of the log information included in the status information on the image data groups of the case file. With this configuration, when the same image as the previous observation is to be searched for, it is possible to reduce a download time of the image data.

At Step S317, the estimating unit 862 determines whether the user has accessed all the image data groups of the case file, on the basis of the log information included in the status information on the image data groups of the case file. If the estimating unit 862 determines that the user has accessed all the image data groups of the case file (Step S317: Yes), the terminal device 8 proceeds to

Step S315 to be described later. In contrast, if the estimating unit 862 determines that the user has not accessed all the image data groups of the case file (Step S317: No), the terminal device 8 proceeds to Step S318 to be described later.

Step S318 and Step S319 respectively correspond to Step S111 and Step S112 in FIG. 5 as described above. Further, Step S320 to Step S324 respectively correspond to Step S118 to Step S122 in FIG. 5 as described above. After Step S324, the terminal device 8 terminates the process.

According to the third embodiment described above, it is possible to reduce a download wait time at the time of observation. Further, the transmission image data groups are set while excluding an image data group that is determined as being not needed for observation, so that it is possible to prevent download of unnecessary image data. As a result, it is possible to reduce the communication traffic, so that it is possible to prevent an increase in a communication cost.

Furthermore, in the third embodiment, it may be possible to estimate a type of the observation workflow on the basis of the presence/absence information related to presence or absence of each of the log information, the finding information that is related to findings of the user on the image data groups, the diagnosis information that is based on an observation result on the image data groups, and the signature information that is related to a signature of the user who has given the diagnosis information, with respect to the status information on the image data groups of the case file.

### Modification of Third Embodiment

Next, a modification of the third embodiment will be described. A terminal device according to the modification of the third embodiment has the same configuration as the terminal device 8 according to the third embodiment as described above, but performs a different process. In the following, a process performed by the terminal device according to the modification of the third embodiment will be described. The same components as those of the endoscope system 1 according to the third embodiment as described above are denoted by the same reference sings, and explanation thereof will be omitted.

Processes performed by Terminal Device

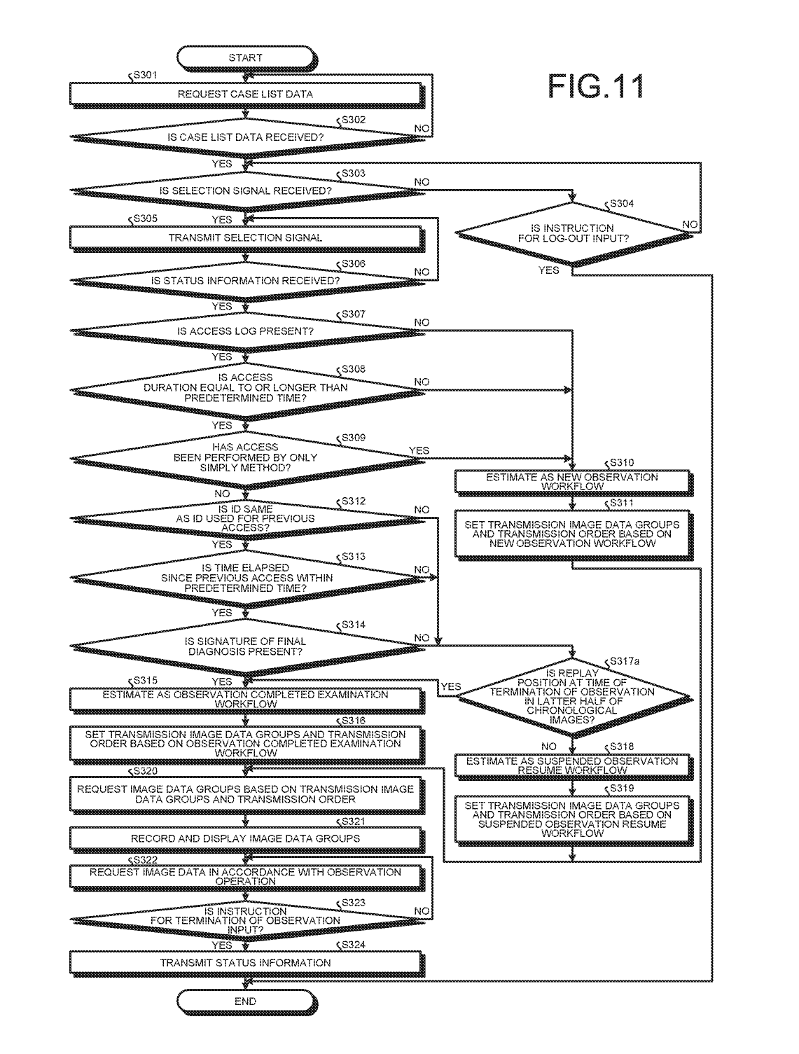

FIG. 11 is a flowchart illustrating the outline of a process performed by the terminal device 8 according to the modification of the third embodiment. The terminal device 8 according to the modification of the third embodiment performs Step S317a instead of Step S317 in FIG. 9 as described above. The other processes are the same as those of FIG. 9 as described above, and therefore, explanation thereof will be omitted.

At Step S317a, the estimating unit 862 determines whether a replay position at the time of termination of observation is in a latter half of chronological images, on the basis of the log information on the image data groups of the case file. Specifically, the estimating unit 862 determines whether a replay position at the time of termination of observation by the user, that is, the position of the slide bar T2 (see FIG. 4), is in a latter half of the chronological images, on the basis of the operation log information of the log information on the image data groups of the case file. It is of course possible for the estimating unit 862 to determine whether the position is in the latter half of the chronological images depending on the time of the slide bar T2 or the time of the time code T3, on the basis of the log information on the image data groups of the case file. If the estimating unit 862 estimates that the replay position at the time of termination of observation is in the latter half of the chronological images (Step S317a: Yes), the terminal device 8 proceeds to Step S315 to be described later. In contrast, if the estimating unit 862 estimates that the replay position at the time of termination of observation is not in the latter half of the chronological images (Step S317a: No), the terminal device 8 proceeds to

Step S318.

According to the modification of the third embodiment described above, it is possible to reduce a download wait time at the time of observation. Further, the transmission image data groups are set while excluding an image data group that is determined as being not needed for observation, so that it is possible to prevent download of unnecessary image data. As a result, it is possible to reduce the communication traffic, so that it is possible to prevent an increase in a communication cost.

### Fourth Embodiment

Next, a fourth embodiment will be described. In an endoscope system according to the fourth embodiment, a server is configured differently from the server 7 of the endoscope system 1 according to the first embodiment as described above. Further, the terminal device 8 of the first embodiment sets image data groups to be transmitted from the server 7 and a transmission order and issues a request to the server 7; however, in the fourth embodiment, the server sets image data groups to be transmitted to the terminal device and a transmission order and transmits them to the terminal device. In the following, a configuration of the server according to the fourth embodiment is first described, and thereafter, a process performed by the server according to the fourth embodiment will be described. The same components as those of the the endoscope system 1 according to the first embodiment as described above are denoted by the same reference signs, and explanation thereof will be omitted.

Detailed Configuration of Server

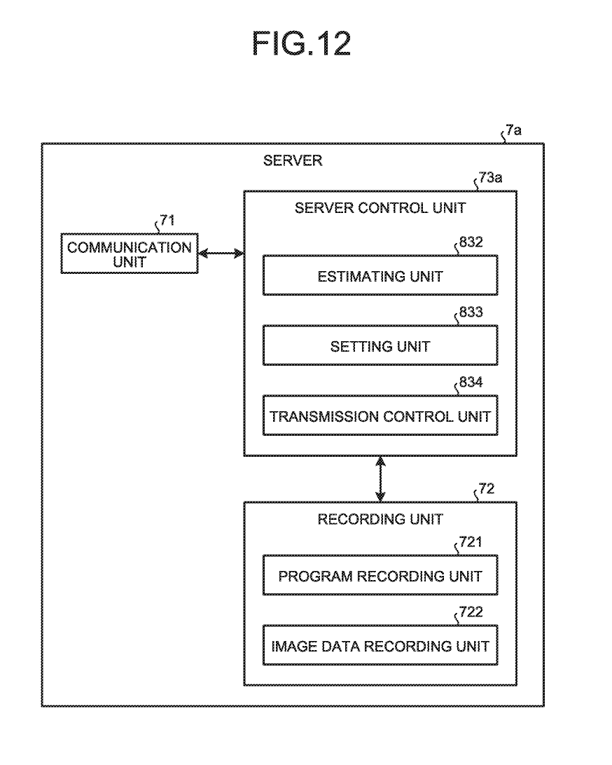

FIG. 12 is a block diagram illustrating a functional configuration of the server according to the fourth embodiment. A server 7a illustrated in FIG. 12 includes a server control unit 73a instead of the server control unit 73 of the server 7 according to the first embodiment as described above.

The server control unit 73a comprehensively controls operation of the server 7a by transferring instructions and data to each of the units included in the server 7a. The server control unit 73a includes an estimating unit 832, a setting unit 833, and a transmission control unit 834.

The estimating unit 832 estimates an observation workflow for browsing image data groups on the terminal device 8, on the basis of the status information indicating a status of a case file selected by the terminal device 8.

The setting unit 833 sets transmission image data groups that are to be transmitted to to the terminal device 8 and a transmission order with respect to the image data groups of the case file selected by the terminal device 8, on the basis of the observation workflow estimated by the estimating unit 832.

The transmission control unit 834 controls sequential transmission of the transmission image data groups to the terminal device in accordance with the transmission order set by the setting unit 833.

Processes performed by Server

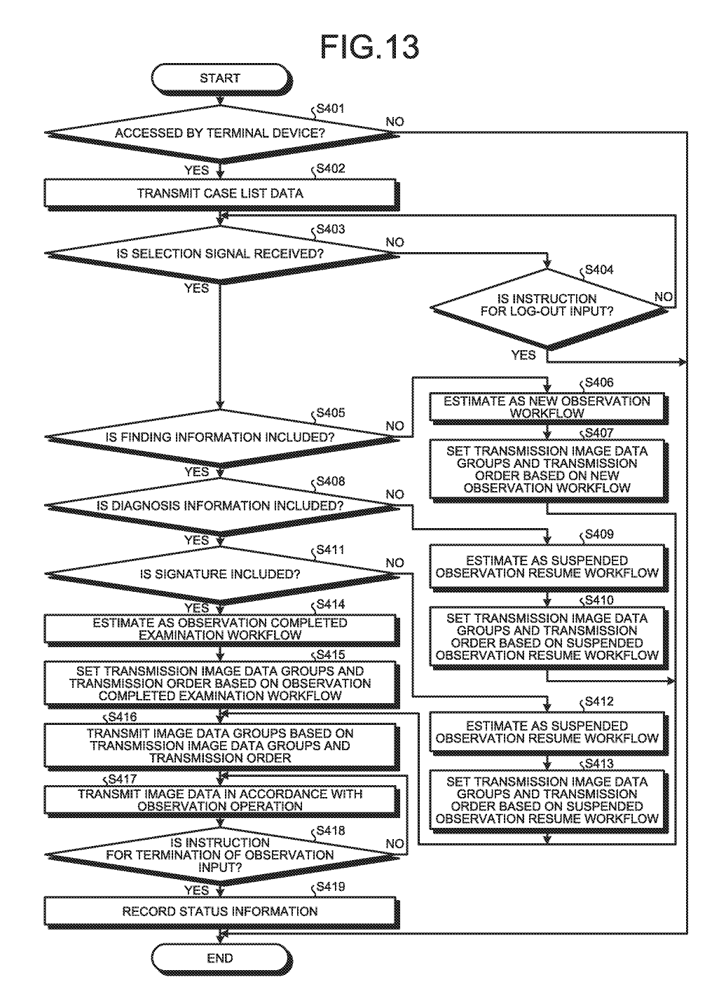

Next, a process performed by the server 7a will be described. FIG. 13 is a flowchart illustrating the outline of the process performed by the server 7a.

As illustrated in FIG. 13, when accessed by the terminal device 8 via the network N100 (Step S401: Yes), the server 7a transmits case list data for each of case files recorded in the recording unit 72 to the terminal device 8 that has accessed the server 7a (Step S402).

Subsequently, when a selection signal (request signal) for selecting any one of a plurality of cases recorded in the recording unit 72 is received from the terminal device 8 via the network N100 (Step S403: Yes), the server 7a proceeds to Step S405 to be described later. In contrast, when the selection signal for selecting any one of the plurality of cases recorded in the recording unit 72 is not received from the terminal device 8 via the network N100 (Step S403: No), the server 7a proceeds to Step S404 to be described below.

Step S404 to Step S415 respectively correspond to Step S104 and Step S107 to Step S117 in FIG. 5 as described above, and the estimating unit 832 and the setting unit 833 of the server 7a instead of the estimating unit 862 and the setting unit 863 of the terminal device 8 perform the same processes; therefore, detailed explanation thereof will be omitted.

At Step S416, the transmission control unit 834 transmits the image data groups to the terminal device 8 via the network N100, on the basis of the transmission image data groups and the transmission order of the case file set by the setting unit 833.

Subsequently, the transmission control unit 834 transmits image data via the network N100 in accordance with observation operation that is performed by the user via the operation input unit 84 of the terminal device 8 (Step S417).

Thereafter, if a termination signal for designating termination of observation is input from the operation input unit 84 of the terminal device 8 via the network N100 (Step S418: Yes), operation status information related to an operation history of the observation that is performed by the terminal device 8 on the case file is recorded in the recording unit 72 in association with the status information on the case file (Step S419). After Step S419, the server 7a terminates the process. In contrast, if the termination signal for designating termination of observation is not input from the operation input unit 84 of the terminal device 8 via the network N100 (Step S418: No), the server 7a returns to Step S417.

At Step S401, when not accessed by the terminal device 8 via the network N100 (Step S401: No), the server 7a terminates the process.

According to the fourth embodiment described above, it is possible to reduce a download wait time at the time of observation in the terminal device 8. Further, the transmission image data groups are set while excluding an image data group that is determined as being not need for observation, so that it is possible to prevent download of unnecessary image data. As a result, it is possible to reduce the communication traffic of the terminal device 8, so that it is possible to prevent an increase in a communication cost.

Furthermore, in the fourth embodiment, a type of the observation workflow is estimated on the basis of the presence/absence information related to presence or absence of each of the finding information that is related to findings of the user on the image data groups, the diagnosis information that is based on an observation result on the image data groups, and the signature information that is related to a signature of the user who has given the diagnosis information, with respect to the status information on the image data groups of the case file selected by the terminal device 8. However, similarly to the third embodiment as described above, it may be possible to estimate a type of the observation workflow on the basis of the log information included in the status information on the image data groups of the case file selected by the terminal device 8. It is of course possible to estimate a type of the observation workflow on the basis of the presence/absence information related to presence or absence of each of the finding information that is related to findings of the user on the image data group, the diagnosis information that is based on an observation result on the image data group, and the signature information that is related to a signature of the user who has given the diagnosis information, and on the basis of the log information, with respect to the status information on the image data groups of the case file selected by the

### Other Embodiments

The present disclosure is not limited to the embodiments as described above, and various modifications and applications are of course applicable within the scope of the gist of the present disclosure. For example, apart from the endoscope system used in the description of the present disclosure, the technology is applicable to a server that distributes moving images to a mobile phone, a smartphone, and the like via a network, a network attached storage, a video camera, an endoscope, a monitoring camera, an imaging apparatus capable of performing bi-directional communication with a network via an optical equipment, such as a microscope, and the like.

Furthermore, while a terminal device downloads image data groups from a single server in the present disclosure, the technology is applicable to a case where image data groups are downloaded from a plurality of servers.

Moreover, in describing the operational flowcharts in this specification, operations are described using expressions, such as “first”, “next”, “subsequently”, and “thereafter”, for the sake of convenience, but the sequences of the operations need not always be performed in this order.

Furthermore, the methods of the processes performed by the terminal device and the server in the embodiments described above, that is, the processes described in the flowcharts, may be stored as programs that are executable by a control unit, such as a CPU. In addition, the programs may be distributed by being stored in a storage medium of an external storage device, such as a memory card (a ROM card, a RAM card, or the like), a magnetic disk, an optical disk (a CD-ROM, a DVD, or the like), or a semiconductor memory. Then, the control unit, such as a CPU, reads the programs stored in the storage medium of the external storage device, and controls operations by the read programs, to thereby be able to perform the processes as described above.

According to the present disclosure, it is possible to effectively transmit image data groups.

In the specification or the drawings, a term that is at least once described together with a different term having a broader meaning or the same meaning may be replaced with the different term at any point in the specification or the drawings. Thus, various modifications and applications may be made without departing from the scope of the disclosure.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
a server configured to accumulate image data groups and status information that includes operation history of observation corresponding to each of the image data groups;
a terminal device configured to allow selecting any of the image data groups accumulated in the server via a network, the terminal device allowing browsing the selected image data groups; and
a processor comprising hardware that is provided in the server or the terminal device, wherein the processor is configured to execute:
estimating a type of an observation workflow that differs depending on whether observation operation has been performed on the selected image data groups or whether the performed observation operation has been completed, the estimation being performed on the basis of status information corresponding to the selected image data groups;
setting transmission image data groups being images to be transmitted from the server to the terminal device among the selected image data groups on the basis of the estimated type of the observation workflow; and
performing control of transmitting the set transmission image data groups from the server to the terminal device.

2. The endoscope system according to claim 1, wherein the status information includes presence/absence information related to presence or absence of each of finding information that is related to findings of a user on the image data groups, diagnosis information that is related to diagnosis based on an observation result on the image data groups, and signature information that is related to a signature of a user who has written the diagnosis information.

3. The endoscope system according to claim 1, wherein the status information includes access log information on access to the image data groups by the terminal device, operation log information at a time of browsing the image data groups by the terminal device, browsing mode log information on a browsing mode at a time of browsing the image data groups by the terminal device, identification log information for identifying a user who has performed observation on the image data groups, and browsing time log information on a browsing time at a time of browsing the image data groups by the terminal device.

4. The endoscope system according to claim 1, wherein the processor sets a transmission order of transmitting the selected image data groups to the terminal device on the basis of the estimated type of the observation workflow.

5. The endoscope system according to claim 1, wherein,
when the processor estimates the type of the observation workflow as a new observation workflow for performing new observation on the image data groups, the transmission image data groups are set such that an entire image of the selected image data groups can be viewed from a higher perspective among the selected image data groups,
when the processor estimates the type of the observation workflow as a suspended observation resume workflow for suspending observation on the image data groups and thereafter resuming the observation, the transmission image data groups are set with reference to an imaging time of image data that is present immediately before the observation is suspended among the selected image data group, and
when the processor estimates the type of the observation workflow as an observation completed examination workflow for performing observation again after observation is completed, only image data to which predetermined information is added is set as the transmission image data groups among the selected image data groups.

6. The endoscope system according to claim 1, wherein, when the processor designates the type of the observation workflow as an additional new observation workflow for additionally performing new observation among the selected image data groups, the processor sets the transmission image data groups such that an entire image of the selected image data groups can be viewed from a higher perspective among the selected image data group.

7. The endoscope system according to claim 1, wherein the processor sets the transmission image data groups while excluding an image data group that is determined as being not needed to be transmitted in previous observation among the selected image data groups.

8. The endoscope system according to claim 2, wherein the processor
estimates the type of the observation workflow as a new observation workflow for performing new observation on the image data groups when the finding information is absent,
estimates the type of the observation workflow as a suspended observation resume workflow for suspending observation on the image data groups and thereafter resuming the observation when the finding information is present and each of the diagnosis information and the signature information is absent, and
estimates the type of the observation workflow as an observation completed examination workflow for performing observation again after observation is completed when each of the finding information, the diagnosis information, and the signature information is present.

9. The endoscope system according to claim 1, wherein the terminal device includes:
a temporary recording device configured to temporarily record the transmission image data groups received from the server, and
a display device configured to display images corresponding to the transmission image data groups recorded by the temporary recording device.

10. The endoscope system according to claim 1, wherein, when a termination signal for designating termination of observation is input, the terminal device transmits, to the server, the status information including a history of operation performed on the image data groups by the terminal device.

11. The endoscope system according to claim 1, wherein the network is a wireless communication network.

12. A terminal device capable of performing bi-directional communication with a server that accumulates image data groups and status information that includes operation history of observation corresponding to each of the image data groups, the terminal device allowing browsing images input from the server, the terminal device comprising:
an operation input device configured to receive input of a selection signal for selecting any of the image data groups accumulated in the server; and
a processor comprising hardware, wherein the processor is configured to execute:
estimating a type of an observation workflow that differs depending on whether observation operation has been performed on the selected image data groups or whether the performed observation operation has been completed, the estimation being performed on the basis of status information corresponding to the selected image data groups;
setting transmission image data groups being images to be transmitted from the server to the terminal device among the selected image data groups on the basis of the estimated type of the observation workflow; and
performing control of transmitting the set transmission image data groups from the server to the terminal device.

13. A server capable of performing bi-directional communication with a terminal device that includes a display device capable of displaying an image and an operation input device capable of receiving input of an operation signal for designating various kinds of operation, the server comprising:
a recording device configured to accumulate image data groups and status information that includes operation history of observation corresponding to each of the image data groups; and
a processor comprising hardware, wherein the processor is configured to execute:
estimating, when a request signal is received, which indicates a request of transmission of any one of the image data groups accumulated in the recording device from the terminal device, a type of an observation workflow that differs depending on whether observation operation has been performed on the selected image data groups or whether the performed observation operation has been completed, the estimation being performed on the basis of status information corresponding to the image data group corresponding to the request signal;
setting transmission image data groups being images to be transmitted from the recording device to the terminal device among the image data group corresponding to the request signal on the basis of the estimated type of the observation workflow; and
performing control of transmitting the set transmission image data group from the recording device to the terminal device.

14. A transmission method performed by an endoscope system including a server configured to accumulate image data groups and status information that includes operation history of observation corresponding to each of the image data groups and a terminal device allowing browsing images input via the server, the transmission method comprising:
receiving input of a selection signal for selecting any of the image data groups accumulated in the server via a network;
estimating a type of an observation workflow that differs depending on whether observation operation has been performed on the selected image data groups or whether the performed observation operation has been completed, the estimation being performed on the basis of status information indicating a status of the selected image data groups;
setting transmission image data groups being images to be transmitted from the server to the terminal device among the selected image data groups on the basis of the estimated type of the observation workflow; and
performing control of transmitting the set transmission image data groups from the server to the terminal device.

15. A non-transitory computer-readable recording medium on which an executable program is recorded, the program instructing a processor of an endoscope system, which includes a server configured to accumulate image data groups and status information that includes operation history of observation corresponding to each of the image data groups and a terminal device allowing browsing images input via the server, to execute:
receiving input of a selection signal for selecting any of the image data groups accumulated in the server via a network;
estimating a type of an observation workflow that differs depending on whether observation operation has been performed on the selected image data groups or whether the performed observation operation has been completed, the estimation being performed on the basis of status information indicating a status of the selected image data groups;
setting transmission image data groups being images to be transmitted from the server to the terminal device among the selected image data groups on the basis of the estimated type of the observation workflow; and
performing control of transmitting the set transmission image data groups from the server to the terminal device.
